(19) United States
(12) Patent Application Publication
(10) Pub. No.: US 2005/0186669 A1
(43) Pub. Date: Aug. 25, 2005

(54) Apparatus and method for preparing and culturing cells

(75) Inventors: Lewis Ho, Hsinchu (TW); Yu-Chi Wang, Hsinchu (TW); King-Ming Chang, Hsinchu (TW)

Correspondence Address: BIRCH STEWART KOLASCH & BIRCH, PO BOX 747, FALLS CHURCH, VA, 22040-0747, US

(73) Assignee: CESCO Bioengineering Co., Ltd.

(21) Appl. No.: 11/059,523

(22) Filed: Feb. 17, 2005

Related U.S. Application Data
(60) Provisional application No. 60/546,301, filed on Feb. 20, 2004.

(51) Int. Cl. C12M 1/34

(52) U.S. Cl. 435/287.1

(57) ABSTRACT

An apparatus and method for preparing and culturing cells includes a chamber having an inlet and an outlet for introducing culture medium and air. A cell growth substrate placed in the chamber for cells anchored or embedded. The preferred embodiment of invention claims a novel process for controlling the platform form to tilt at one end, to maintain the seesaw and rocking movement for cultured process. The seesaw movement or rocking movement efficiently the nutrition, carbon dioxide and oxygen transfer during cultured process for production of cellular protein products or harvesting the cells.

### CROSS-REFERENCE TO RELATED APPLICATION

This application claim priority to U.S. provisional 60/546,301 filed Feb. 20, 2004.

### REFERENCE CITED

U.S. Pat. No. 4,334,028

U.S. Pat. No. 4,851,351

U.S. Pat. No. 4,962,033

U.S. Pat. No. 5,010,013

U.S. Pat. No. 5,449,617

U.S. Pat. No. 5,523,228

U.S. Pat. No. 5,527,705

U.S. Pat. No. 5,702,941

U.S. Pat. No. 6,323,022

### BACKGROUND OF THE INVENTION

1. Field of the Invention

The preferred embodiment of invention relates to an apparatus and methods for growing the microorganisms, cells and/or tissue culture in vitro. More particularly, the preferred embodiment of invention relates to a cell culture apparatus containing at least one cell growth substrate which allows rapid and uniform transfer of gases between the environment of cells contained in the cell culture apparatus and the atmosphere of the incubator in which the cell culture apparatus is incubated. The preferred embodiment of invention is more specifically for efficiently control of oxygen and pH during cultural period and maximizing in microorganism, cell and/or tissue culturing of any products produced by the microorganism and/or cellular products, such as protein, and harvesting the same.

2. Background of the Invention

Large-scale cell culture processes have been developed extensively over the years for the growth of bacteria, yeast and molds, all of which typically possess robust cell walls and/or extra cellular materials thus, are more resilient. The structural resilience of these microbial cells is a key factor contributing to the rapidity of the development of highly-efficient cell culture processes for these types of cells. For example, bacterial cells can be grown in very large volumes of liquid medium using vigorous agitation, culture stirring and gas sparging techniques to achieve good aeration during growth, all the while maintaining viable cultures. Alternatively, bacteria can be grown as a biofilm, however, a growing surface would be required.

In contrast, the techniques to culture cells such as eukaryotic cells, animal cells, mammalian cells and/or tissue are more difficult and complex since these cells are more delicate than microbial cells and have nutrient and oxygen requirements during growth which are more complex and difficult to maintain. Further, animal cells and/or mammalian cells cannot withstand the excessive turbulence and/or shear forces that can be created by an influx of air or gaseous mixtures, such as a mixture containing oxygen, nitrogen and carbon dioxide, that are tolerated more easily by microbial cells. In addition, no animal cells can be directly exposed to gases. Most of the animal cells can only utilize dissolved oxygen in the culture medium. Animal cells and mammalian cells are more likely to be damaged by air and gas influx than are microbial cells and thus, result in increased cell mortality. Bioreactors for larger-scale culturing often have internal moving parts, such as an impeller, which subject the cells to a very high fluid shearing force causing cell damage, sometimes cell death, thus leading to low viability of cultures and as a result reduces protein and/or cell by-product production. Likewise, bioreactors that utilize other types of mechanical parts, harsh air movement, or abrupt fluid movement as a mechanism to achieve cell suspension and/or proper aeration will likely cause damage to cells and hinder cell and tissue growth, which further leads to a decrease in cell by-product production, such a protein.

A primary function of a bioreactor is for research wherein large numbers of cells are grown to refine the minute quantities of an active material, including but not limited to a protein or antibody that are secreted by cells into the growth medium. Another function of a bioreactor is the scale-up laboratory cell culture processes for commercial purposes to mass-produce the active proteins made by cells and/or tissues. Because of the need to culture eukaryotic cells and/or prokaryotic cells and/or animal cells and/or mammalian cells in the laboratory in large quantities, bioreactors and culturing apparatus has become an important tool in research and in the production of cells for producing active proteins and/or antibodies and/or any cell by-products.

Many methods are known in the art for growing cells in culture, both on large and small scales. For smaller-scale cell culturing, many vessels have been developed over the years. Culture dishes, for example, represent one type of culturing vessel. Culture dishes typically consist of a bottom dish, which contains the growth medium, and a removable cover. Although the removable cover provides a convenient access to the culture, microorganisms as a result of repeating removing the cover during the culturing process often and easily contaminate cells. In fact, contamination is one of the principal challenges to successful cell and tissue culturing techniques.

To overcome contamination with culture dishes, culture flasks were developed. Culture flasks typically have a culture chamber, a small tubular opening located at one end of the flask and a corresponding closure. This design attempts to minimize the exposure of cells to dust, bacteria, yeast and other contaminants. For example, patents teaching culture flasks can be found in U.S. Pat. No. 4,334,028 to Carver and U.S. Pat. No. 4,851,351 to Akamine and U.S. Pat. No. 5,398,837 to Degrassi. Although culture flasks were an improvement over culture dishes, they did not fully remedy the contamination problem. In addition, neither the culture dish nor the culture flask can provide appropriate aeration to cells. Furthermore, the growth surface area available in culture flasks is not adequate, as in culture dishes; thus, placing limits on scaling-up the culturing process using this technology.

Another technology developed for use in cell and tissue culturing were roller bottles. The roller bottle has been widely used in the art for many years. Although they offer some advantages over dishes and flasks, such as a larger surface area for cell attachment and growth, they are still unable to remedy all of the deficiencies and particularly with scaling up. Collectively, these weaknesses include but are not limited to the large uncontrollable hydrodynamic shear forces associated with a gas headspace and the abundance of turbulent eddies. As a result of the high shear force environment inherent in roller bottles, tissue culturing of larger three-dimensional structures is virtually impossible. Only those cell types that are not damaged by the shear forces and/or are capable of remaining adhered to the wall of the roller bottles can be maintained in culture for an extended period of time. Therefore, long-term maintenance of established cell lines can prove to be difficult with roller bottles due to the constant challenge of the high shear force environment and possible contamination. Examples of patents directed to roller bottles include U.S. Pat. No. 5,527,705 to Mussi et al. and U.S. Pat. No. 4,962,033 to Serkes.

Moreover, although the surface area of roller bottles is greater by comparison to culture flasks and dishes, it is often not considered adequate since the surface for cell adhesion is not necessarily more favorable than the culture flasks and dishes, particularly for scaling up the growth of cell cultures. Some efforts have been made to improve upon the roller bottle by providing a greater amount of surface area per roller bottle. For example, U.S. Pat. No. 5,010,013 to Serkes describes a roller bottle with increased surface area for cell attachment. Serkes relates to the use of corrugated channels added to the interior surface area of the roller bottle to increase capacity for cellular attachment. However, a typical roller bottle provides only a surface area of about 850-1700 cm² for cultivating cells, a multitude of roller bottles are still required for scaling up production. Although, automation of culturing with a large plurality of roller bottles can save on time and labor investment, these operations are typically costly and limiting.

In addition to the problems of hydrodynamic shear forces and surface-area limitations, a central problem inherent in cell and tissue culturing techniques is attaining and maintaining sufficient oxygenation in the growing culture. It is well-known in the art that prokaryotic cells, eukaryotic cells, including animal cells, mammalian cells, insect cells, yeast and molds all have one major rate-limiting step, oxygen mass transfer.

Oxygen metabolism is essential for metabolic function of most prokaryotic cells and eukaryotic cells with the exception of some fermentative-type metabolisms of various eukaryotic microorganisms, such as yeast. Particularly, with mammalian and animal cell culturing techniques, oxygen flux is especially important during the early stages of rapid cell division. Oxygen utilization per cell is greatest when cells are suspended; requirements for oxygen decrease as the cells aggregate and differentiate. Some mammalian and animal cells are anchorage-dependent, requiring a surface to grow, whereas other mammalian and animal cells are anchorage independent and can be grown in liquid environments regardless of the types of cells. However, these cells all require dissolved oxygen in the medium. Nevertheless, during the later phases of cell culture with both anchorage-dependent and independent cells, as the number of cells per unit volume increases, the bulk oxygen mass transfer requirement once again increases.

Traditionally, at least with anchorage-independent cells, increased requirements for oxygen are accommodated by mechanical stirring methods and the sparging of gases into the culture. However, as discussed, both stirring and the sparging of gases can result in damaging cells, thereby decreasing the viability of the culture and the overall efficiency and productivity of the cell and/or tissue culture. Further, direct sparging of cell and tissue cultures with gas can lead to foam production which, is also detrimental to cell viability.

Some attempts have been made in the art to solve the oxygenation problem during cell culturing. For example, U.S. Pat. No. 5,153,131, issued to Wolf et al. (“Wolf”), relates to a bioreactor vessel without mixing blades. Instead, air travels through an air inlet passageway through a support plate member across a screen and through a flat disk permeable membrane wedged between the two sides of the vessel housing. The oxygen then diffuses across the membrane into the culture chamber due to the concentration gradient between the two sides of the housing.

The Wolf bioreactor, however, presents many disadvantages. Particularly, the rate at which oxygen can diffuse across the disk-shaped membrane is a significant limitation that restricts the size of the culture chamber. Another disadvantage of the flat disk membrane is that it is designed to flex in order to cause mixing within the culture chamber, which can result in cell death. The mixing effect is a feature described as being critical for the distribution of air throughout the culture media, however, it will also tend to create shear forces within the chamber, again can be detrimental to cells, consequently providing sufficient gas exchange to sustain the growth of larger cellular structures is a significant and realistic restriction when designing a bioreactor or culture vessel.

An example showing an attempt to overcome the deficiencies thus far described is to make reactors from gas permeable materials. For instance, U.S. Pat. No. 5,702,941, issued to Schwarz et al. (“Schwarz”), entitled “Gas Permeable Bioreactor And Method Of Use” relates to a vessel that is horizontally rotated and the vessel is at least partially composed of gas permeable materials. The gas exchange with the culture medium is intended to occur directly through the gas permeable materials of which the vessel walls are composed.

However, Schwarz discloses that the range of sizes for the vessel is still limited since gas exchange is dependent on the quantity of gas permeable surface area. Schwarz emphasized that as the surface area of the vessel increases, the volume and the amount of culture medium also increases. As such, the preferred dimensions of the vessel described in Schwarz are limited to between one and six inches in diameter while the width is, according to Schwarz, preferably limited to between one-quarter of one inch and one inch. Such size limitations are not suitable for growing three-dimensional cellular aggregates and tissues and/or any scaling up production.

Similarly, U.S. Pat. No. 5,449,617, issued to Falkenberg et al. (“Falkenberg”), entitled “Culture Vessel For Cell Culture” relates to a vessel that is horizontally rotated. The vessel is divided by a dialysis membrane into a cell culturing chamber and a nutrient medium reservoir. Gas permeable materials are used in the vessel walls to enable gas exchange in the cell culturing chamber. However, the vessel is not completely filled with the nutrient medium and a large volume of air is maintained above the fluid medium in both chambers. The Falkenberg vessel, however, is not designed to minimize turbulence within the cell culture chamber but rather, mixing is recited to be an essential step to keep the dialysis membrane wetted. Further, Falkenberg does not contemplate using the vessel to grow cellular aggregates or tissues of any kind.

Another solution has been to develop flexible, disposable plastic vessels that do not require cleaning or sterilization and require only minimal validation efforts. For example, U.S. Pat. No. 5,523,228 describes a flexible, disposable, and gas permeable cell culture chamber that is horizontally rotated. The cell culture chamber is made of two sheets of plastic fused together. The edges of the chamber, beyond the seams, serve as points of attachment to a horizontally rotating drive means. The cell culture chamber is made of gas permeable material and is mounted on a horizontally rotating disk drive that will support the flexible cell culture chamber without blocking airflow over the membrane surfaces. Thus, the cell culture chamber is placed in an incubator and oxygen transfer controlled by controlling the gas pressure in the incubator according to the permeability coefficient of the bag. The rotation of the bag assists in mixing the contents of the bag and enhances gas transfer throughout the bag. However, the cell culture chamber is limited to use within a controlled gas environment. Furthermore, the cell culture chamber has no support apparatus and is therefore limited to small volumes. The described cell culture chamber is actually a batch culture apparatus in that it does not provide an inlet and an outlet for media to be constantly pumped into and out of the chamber during rotation.

Wave Biotech (Bridgewater, N.J.) has also developed a range of pre-sterile, disposable bioreactors that do not require cleaning or sterilizing. The Wave Bioreactor is made of sheets of flexible, gas impermeable material. The bag is partially filled with media and then inflated with air that continually passes through the bag's headspace. The media is mixed and aerated by rocking the bags up to 40 times a minute to increase the air-liquid interface. However, since a solid housing does not support the bags, the bags become unwieldy and difficult to handle as they increase in size. Furthermore, the wave action within the rocking bag creates damaging turbulent forces. Certain cell cultures, particularly human cell cultures, thrive better under more gentle conditions.

There is a continuing need to develop lightweight, presterilized, disposable bioreactors with simple connections to existing equipment that require little training to operate, yet provide the necessary gas transfer and nutrient mixing required for successful cell cultures.

Given the importance of cell and tissue culture technology in biotechnology research, pharmaceutical research, patient care, academic research and in view of the deficiencies, obstacles and limitations exist in the prior art described the preferred embodiment of invention overcomes the obstacle and remedies the deficiencies in the prior art by teaching and disclosing a method and an apparatus for cell and tissue culturing that fulfills the long-felt need for a novel method and apparatus to culture cells and tissues that is more reliable, less complex, more efficient, less cumbersome, less expensive, less-labor intensive, capable of increasing culture scale with no limitation of oxygen supply, capable of increasing cell vitality and producing a higher yield of cellular by-products generated from the cells.

### SUMMARY OF THE INVENTION

There are several obstacles remain unsolved in the cell culture technology. One of the greatest obstacles in cell and/or tissue culturing is that the apparatuses and/or apparatus and/or method employed are difficult to strike a balance between providing enough oxygen but still can avoid injuring the cells. Another obstacle in mammalian cell culture is that a relative large amount of inoculum is required to initiate a culture. Therefore, it makes the scale up issue very difficult.

The invention comprehends both the apparatus and the method for use it. The apparatus for cell-culturing having features of the preferred embodiment of invention comprises of a chamber which contain a inlet and/or a outlet, a cell growth substrate placing in a chamber, the chamber which is secured on the platform, a fulcrum under the platform; a power plant connecting the fulcrum to move the platform in a seesaw motion or rocking motion in a single or two degree of freedom; a timing controller controlling the platform to tilt at one end and keep for a period of time and keep for a cultured period of time.

The method for cell-culturing in the preferred embodiment of invention comprise the steps of preparing a sterile chamber which has a single hollow interior volume, placing a cell growth substrate in the chamber, introducing air containing oxygen using the inlet to inflate the said chamber, introducing cell culture medium using a inlet, placing cells suspension into the chamber to distribute cells on the cell growth substrate, securing the chamber on the platform, placing a fulcrum under the platform, introducing air containing oxygen using a inlet, and opening a outlet, inflating chamber with air continual pass through chamber, connecting a fulcrum with power plant to move the platform in a seesaw or rocking motion, setting up a timing control in order to keep one of cell culture substrate be submerged or exposed for a period of time in order to allow cells to anchor on the cell growth substrate or be embedded in the cell growth substrate, keeping the seesaw or rocking motion until cells are anchored on growth substrate and/or embedded the cell growth substrate, setting up a timing control keeping one of cell culture substrate be submerged or exposed for a period of time in order to complete the nutrient exchange or control nutrient supply for both gaseous and liquid phase, keeping moving the platform in a seesaw or rocking motion wherein the cell culture substrate being submerged at one side whereby the necessary carbon dioxide and nutrition being transferred/mixed and thin film of culture medium exposed on culture substrate at the other side whereby oxygen is received through a thin film medium interface without directly contacting air during the see-saw or rocking motion, periodically removing conditioned culture medium wherein containing cellular products secreted by cells , periodically repeating above steps from setting up timing control to the end process for maintaining suitable continual cell culture condition during the cultured period.

The apparatus and the method in the preferred embodiment of invention providing a novel method for the culturing of cells in resolving the greatest obstacles about effectively oxygen/nutrient transfer, minimal metabolite waste accumulation, air bubbles and/or shear forces caused by an infusion of gases, culturing maximizes cells adhesion, increased surface area for air-medium contact and functions as a static mixer when the medium in the apparatus of the preferred embodiment of invention.

The preferred embodiment of invention provides a reliable, simple, inexpensive and efficient method for culturing cells and/or tissues and for harvesting cellular products produced thereof. such as prokaryotic cells, eukaryotic cells, animal cells, mammalian cells, whereby a continuous supply of both oxygen and nutrients to the cells are provided without directly exposing any cells to gas, thus reduces cell injury and even cell mortality. Moreover, the method of the preferred embodiment of invention reduces waste accumulation by providing sufficient oxygen during culture, helps removing excess carbon dioxide during culture, helps stabilizing culture environment with a simple, reliable, inexpensive and efficient mean, helps preventing detrimental effects on cells caused by air bubbles and gases. Moreover, the method of the preferred embodiment of invention could reduce initial seeding density that are usually required in animal cell culture, and also eliminate lag phase during initial growth period originally due to low inoculum density. Moreover, the method of the preferred embodiment of invention teaches and discloses a novel and simple mean for nutrient control similar to traditional fed-batch process; whereby, metabolism could be properly controlled during growth and production phases. Moreover, the preferred embodiment of invention teaches and discloses a novel method for efficiently removing carbon dioxide and stabilizing pH during culture. Furthermore, the preferred embodiment of invention provides a method for an easier and more convenient way to produce and harvest secreted cellular products, such as protein, and/or antibiotics, and/or any cellular and/or tissue products from cell or tissue cultures.

These and other embodiments are disclosed or are obvious from and encompassed by the following Detailed Description.

### BRIEF DESCRIPTION OF THE DRAWINGS

The following FIGS are given for the purpose of illustration only and are not intended to limit the scope of the preferred embodiment of invention.

FIG. 1 shows a stereograph of a cell-cultivating apparatus of the preferred embodiment of invention wherein the growth substrate means are disposed at the first end and the second end of the bag chamber. Bag chamber is inflated by continuous inflow of outflow of air. The culture medium is partially filled in the bag. The bag chamber is mounted on a tray connected by a driving means.

FIG. 2 shows a side view of a cell-cultivating apparatus of the preferred embodiment of invention wherein the growth substrate means are disposed at the first end and the second end of the bag chamber. Bag chamber is inflated by continuous inflow of outflow of air. The culture medium is partially filled in the bag. The bag chamber is mounted on a tray connected by a driving means.

FIG. 3 shows the mechanism of a cell-cultivating apparatus of the preferred embodiment of invention where in the bag is tilted to an angle by the driving means in order to lower one end of the bag chamber. A power plant connecting to fulcrum to move the platform in a seesaw or rocking motion culture medium flow to the other end of the chamber. The growth substrate means in the higher end of the bag chamber is thus emerge from the culture medium and exposed to the gaseous environment, while the growth substrate means in the lower end of the bag chamber is thus submerge to the culture medium.

FIG. 4 shows the mechanism of a cell-cultivating apparatus of the preferred embodiment of invention where in the controller switch the tray position and causing the bag chamber to be tilted to the other side with an angle in order to lower the other end of the bag chamber. A power plant connecting to fulcrum to move the platform in a seesaw or rocking motion culture medium flow to the other end of the chamber. The growth substrate means in the higher end of the chamber is thus emerge from the culture medium and exposed to the gaseous environment, while the growth substrate means in the lower end of the bag chamber is thus submerge to the culture medium.

FIG. 5 shows a cell-cultivating apparatus of the preferred embodiment of invention wherein the growth substrate means is disposed at the first end or at the second end of the bag chamber. The bag chamber is mounted on a tray connected by a driving means. The inlet air inflates bag chamber. The culture medium is partially filled in the bag. When the bag is tilted to an angle in order to lower one end of the bag chamber, the culture medium flow to the lower end of the bag chamber by power plant connecting to fulcrum to move the platform in a seesaw or rocking motion. The growth substrate means in the lower end of the bag chamber is thus submerge to the culture medium. When the controller switch its position and causing the bag chamber to be tilted to the other side with an angle in order to lower the other end of the bag chamber, the culture medium flow to the other end of the bag chamber by power plant connecting to fulcrum to move the platform in a seesaw or rocking motion. The growth substrate means in the higher end of the bag chamber is thus emerge from the culture medium and exposed to the gaseous environment.

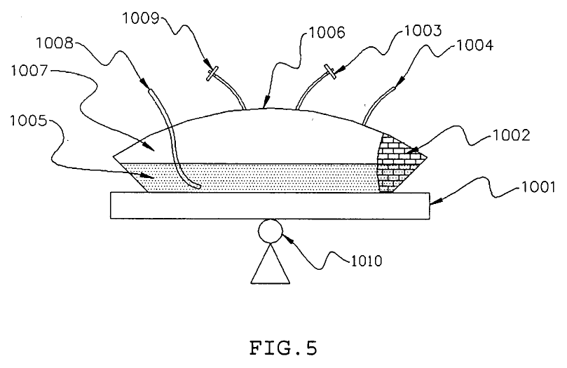

FIG. 6 shows a side view of a cell-cultivating apparatus of the preferred embodiment of invention wherein the growth substrate means are disposed at the first end of the chamber. The culture medium is partially filled in the bag. The bag chamber is mounted on a tray connected by a driving means.

FIG. 7 shows the mechanism of a cell-cultivating apparatus of the preferred embodiment of invention where in the chamber is tilted to an angle in order to lower one end of the bag chamber, the culture medium flow to the lower end of the bag chamber by power plant connecting to fulcrum to move the platform in a seesaw or rocking motion. The growth substrate means in the higher end of the bag chamber is thus emerged from the culture medium and exposed to the gaseous environment, while the growth substrate means in the lower end of the bag chamber is thus submerge to the culture medium.

FIG. 8 shows the mechanism of a cell-cultivating apparatus of the preferred embodiment of invention where in the controller switch the tray position and the chamber is tilted to the other side with an angle in order to lower the other end of the bag chamber, the culture medium flow to the other end of the chamber by power plant connecting to fulcrum to move the platform in a seesaw or rocking motion. The growth substrate means in the higher end of the chamber is thus emerge from the culture medium and exposed to the gaseous environment, while the growth substrate means in the lower end of the chamber is thus submerge to the culture medium.

FIG. 9. illustrates Distribution of Mouse hybridoma (OKT3) cells anchored on cell growth substrate described in Example 1.

FIG.10. illustrates Distribution of Mouse hybridoma (OKT3) cells anchored on cell growth substrate described in Example 1.

FIG. 11. illustrates Compare Glucose uptake rate and Glutamine uptake rate (GUR )between the referred embodiment of invention and BelloCell described in Example 2.

FIG. 12. illustrates Compare L/G with pH value between present invention and BelloCell described in Example 3.

FIG. 13. illustrates The photo of the preferred embodiment of invention described in Example 4.

### DETAILED DESCRIPTION OF THE INVENTION

In according with the preferred embodiment of invention, there is provided an apparatus and method for preparing and culturing cells. The embodiments of the preferred embodiment of invention can be used to culture different variety cells, such as eukaryotic and prokaryotic cells, particularly animal cells and/or mammalian cells.

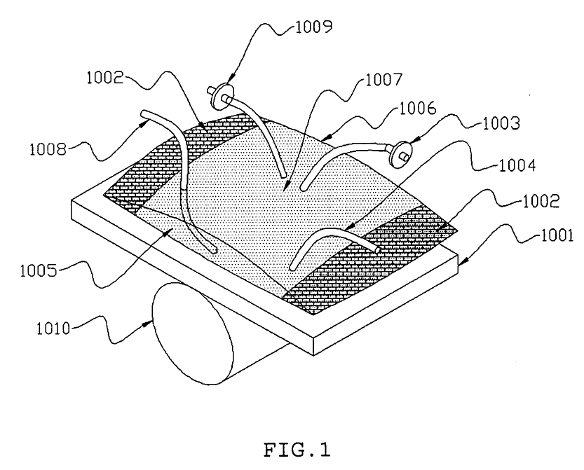

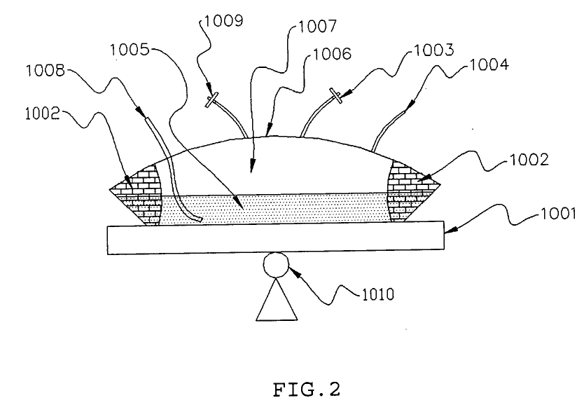

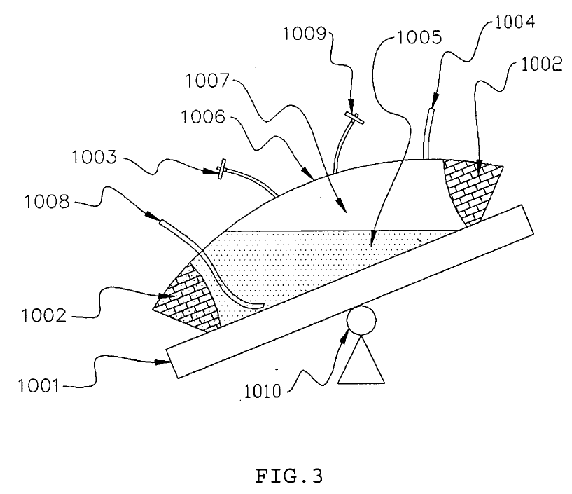

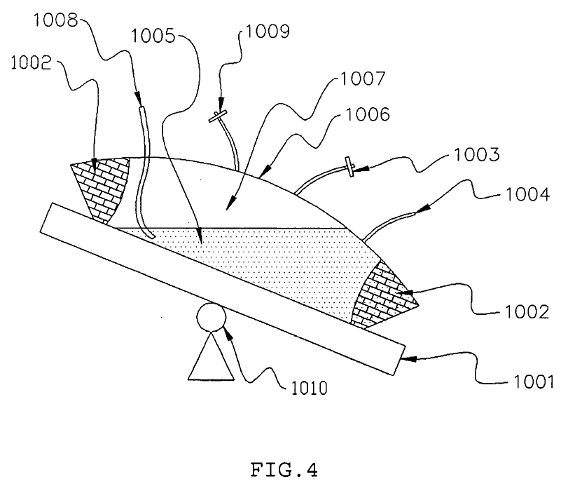

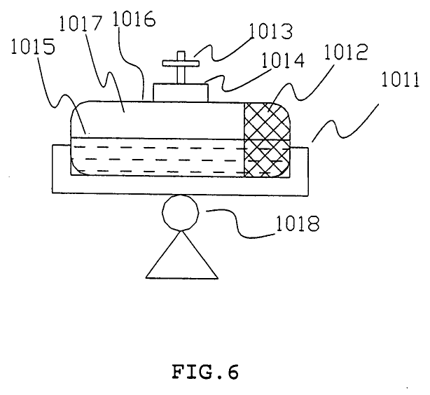

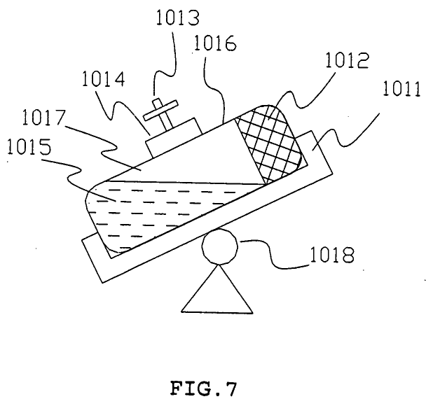

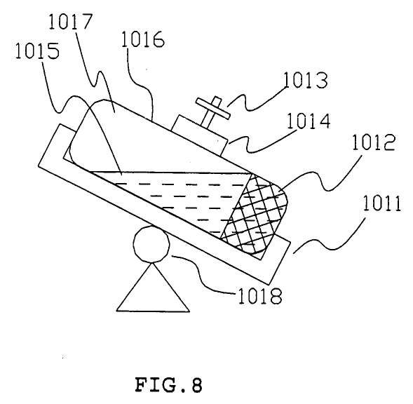

The apparatus for preparing and culturing cells is shown in FIG. 1 (perspective view), FIG. 2 (side view) and FIG. 6 (perspective view). The apparatus includes a pre-sterilized chamber which contain at least one inlet and/or one out let, a cell growth substrate placed in the chamber, a platform, the chamber which is secured on the platform, a fulcrum under the platform, a power plant which connected to the fulcrum to move the platform in a seesaw motion or rocking motion (FIG. 3, FIG. 4, FIG. 7 and FIG. 8.) in single or two degree of freedom, a timing controller.

The chamber is a rigid or flexible plastic bag, which has a single hollow interior space for placing cell growth substrate, culture medium and culturing cells. The chamber, for example, can contain, inter alia, cells of desire, growth medium and a growth substrate means to provide a surface area for cell adhesion and growth. In a preferred embodiment, the chamber is a flexible, plastic bag.

Reference is now made to the figures by way of examples and they are by no way limiting the scope of the preferred embodiment of invention. Referring to FIG. 1 and FIG. 2, a stereograph and side view of the cell-cultivating apparatus of a first embodiment of the preferred embodiment of invention is presented. Reference is made to the figures. A chamber 1006 contains inlet opening 1003, and outlet opening 1009 for air flow in and out, inlet opening 1004, outlet opening 1008 for introduction of culture medium and withdraw of conditioned culture medium, and cell growth substrate means 1002 dispose in two sides of the bag chamber. The inlet 1003 and outlet 1009 opening are further fitted with at least one air filter to sterilize air passing in or out of the cell-cultivating apparatus. The outlet tubing 1008 is further extended into the bottom of the chamber 1006 in order to facilitate the removal of growth medium. The bag chamber 1006 is partially filled with media 1005 through the inlet 1004, and then inflated with air that continually passes through the bag's headspace 1007 from inlet 1003 to outlet 1009. The bag chamber 1006 is mounted and supported by a tray 1001 connected to a driving means 1010 which can alternately tilt, seesaw, or rock the bag chamber 1006 in order to move the liquid from one side of the bag chamber to the other side of the bag chamber.

The cell growth substrate means forms a loosely packed matrix that can function as a depth filter to capture cells during cell plating in order to maximize cell anchored and/or embedded. The cell growth substrate means also maximizes air-medium contact by providing a thin air-growth medium interface when the growth substrate means emerge from the growth medium. The growth substrate means can also function as a static mixer when the growth medium flow through the growth substrate means. The growth substrate means is preferably a porous substrate of any size and shape and can be constructed from any conformation. More particularly, the porous growth substrate means according to the preferred embodiment of invention provides a maximum amount of surface area for cell adhesion, growth, gentle stirring, gentle mixing and oxygenation without ever allowing the cells to come into direct contact with air. The chamber system in accordance with the preferred embodiment of invention also maximizes cell growth by subjecting cells to a periodic and intermittent exposure of nutrients and oxygen. The system in accordance with the preferred embodiment of invention also provides an easy way to collect culture medium containing cellular products as well as replenishing culture medium. The cell-cultivating apparatus of the preferred embodiment of invention also protects cells from being directly exposed to any air, gas bubbles or any shear forces generated by an influx of gas, thus, avoiding any detrimental effects to the cells.

The cell growth substrate placed in the side of the chamber provided for cells anchored and/or embedded. The cell growth substrate is composed of porous material, high-molecular materials, ceramics, fiber, non-woven or woven sheets. The materials are ceramics, polymers, woven substrates, non-woven substrates, polyamide, polyester, polyurethane, fluorocarbon polymers, polyethylene, polypropylene or polyvinyl alcohol, glass. The porous growth substrate means can be made in any form, shape or size including, but not limited to, a disk, flake, block, plate, sheet, strip, pellet, microcarrier, micropellet, macroscopic pellet. The utilization of these growth substrate means in accordance with the preferred embodiment of invention maximizes surface areas for cell adhesion and air-growth medium surface.

The platform can be tilted in a seesaw or rocking movement at a degree from 10 to 180. The frequency of seesaw or rocking movement can make intermittently and periodically movement by cells density and/or different variety cells, but indirectly, exposed to a gaseous environment via a thin gas-growth medium interface in order to receive oxygen or be submerged in culture medium in order to facilitate cell growth and cellular product production.

The chamber, which is secured on the platform, a fulcrum under the platform can control platform to tilt at single or two degree of freedom by power plant with timing controller. In addition, the preferred embodiment of invention is preferably provided with a driving means to control the movement of the culture medium from one end of the chamber to the other end of the chamber in order to facilitate a periodic and intermittent submerging of the cells in growth medium in order to provide needed nutrients and emerging from the growth medium in order to indirectly expose cells to a gaseous environment via a thin gas-growth medium interface to provide sufficient oxygenation. The indirect exposure of the cells to the gaseous environment provides sufficient and efficient oxygenation without harming and/or killing the cells.

When excess culture medium is removed from the substrate means with which cells are immobilized, the cell density on the substrate means is highly concentrated. For example, if there are 500 ml of culture medium cover on 100 ml substrate means and with initial 1×10⁷ cells. The cell density is 2×10⁴ cells/ml. Usually for this cell density, there will have a very long lag phase and will take lots of time to gain enough cell density. When excess culture medium is removed from the substrate means, there will have around 50 ml culture medium remained on the substrate means because of its hydrophilicity. The cell density will then increase to 2×10⁵ cells/ml. This will then bring back the normal initial cell density and will keep the cell growth under normal doubling time. This is due to a concentrated autocrine secreted by cells themselves and triggers the cell cycle for propagation. The substrate means will not dry out due to the substrate means is a hydrophilic material and the culture environment is humidity saturated. When autocrine is accumulated enough to trigger cell propagation, a fresh culture medium can then submerge the substrate means again to exchange waste and nutrient from the conditioned culture medium on the substrate means. The period between emerging and submerging depends on the initial cell density. A lower cell density will require a longer emerging period. For example, if the initial seeding density after emerging is 2×10⁵ cells/ml, it might require a exposing period from several hours to one day; if the initial seeding density after emerging is 1×10⁶ cells/ml, it might require a exposing period from one to several hours.

More particularly, the preferred embodiment of invention is directed to a reliable, simple, inexpensive, disposable, sterile and efficient method for culturing cells and/or tissues and harvesting cellular products produced by cells cultured thereof. More specifically, the preferred embodiment of invention provides a novel method for efficiently culturing any cells whether eukaryotic, prokaryotic, mammalian or animal wherein both oxygen and nutrients needed to ensure cell growth are readily available without causing damage to cells. Furthermore, the method of the preferred embodiment of invention prevent or greatly reduce the metabolite waste accumulation, avoid introducing shear forces on growing cultures, and protect cells from direct exposure to gas, air bubbles and gases. Moreover, the method of the instant invention can either be automated or manually carried out with a minimum level of labor involvement and/or supervision. Further still, the instant invention provides a method for an easier and more convenient means for producing and harvesting secreted cellular products such as proteins, antibodies from cell or tissue cultures.

In another embodiment of the preferred embodiment of invention teaches a cell-cultivating method which can control nutrient supply and promote cell enter secondary production phase. The method comprises the step of: providing a growth substrate to receive cells and allow cell adhesion; providing culture medium to submerge the substrate means to provide sufficient nutrient; removing excess culture medium from the substrate means and remain only a thin film of culture medium covering on the substrate means and replace the space originally occupied by culture medium with gaseous phase; allowing the substrate means to stay in the gaseous phase for a period of time; re-submerging the substrate means with culture medium to provide sufficient nutrient and remove waste from the substrate means; repeat the submerging and emerging step until the purpose of the cell culture is achieved.

When excess culture medium is removed from the substrate means with which cells are immobilized, the nutrient on the substrate means is therefore limited unless the culture medium re-submerges the substrate means again. Therefore, if we control the exposing period, we can control the nutrient availability on the substrate means. Many important biological products such as monoclonal antibody, angiostatin, interferon, . . . are produced by CHO and hybridoma will switch into production phase only if cell growth reach plateau or under slow growth condition. By controlling the nutrient, cell can be teached to enter into production rather in unlimited growth phase. This will then save a lot of time and culture medium.

Carbon dioxide is a metabolic by-product, which is the major source to reduce the pH in culture medium during culture. Because the substrate means will create a large surface area when the excess culture medium is removed and exposed the said substrate means into gaseous phase, it will help to release carbon dioxide from liquid phase into gaseous. For example, during submerge phase, the surface to volume ratio for liquid-gas phase contact is 1, then after emerging phase, the surface to volume ratio for liquid-gas contact will increase to around 200. Therefore, the carbon dioxide release rate will increase several folds. The exposure phase can be ranged between several minutes to several hours unless the nutrient on the substrate means is not enough to support cell growth or activity.

More particularly, the preferred embodiment of invention is directed to a reliable, simple, inexpensive, disposable, sterile and efficient method and apparatus for culturing cells and/or tissues and harvesting cellular products produced by cells cultured thereof. More specifically, the preferred embodiment of invention provides a novel method and apparatus for efficiently culturing any cells whether eukaryotic, prokaryotic, mammalian or animal wherein both oxygen and nutrients needed to ensure cell growth are readily available without causing damage to cells. Furthermore, the method and apparatus of the preferred embodiment of invention prevent or greatly reduce the risk of any type of contamination, avoid introducing shear forces on growing cultures, and protect cells from direct exposure to gas, air bubbles and gases. Moreover, the method and apparatus of the instant invention can either be automated or manually carried out with a minimum level of labor involvement and/or supervision. Further still, the instant invention provides a method and apparatus for an easier and more convenient means for producing and harvesting secreted cellular products such as proteins, antibodies from cell or tissue cultures.

The following detailed description, given by way of example, is not intended to limit the invention to any specific embodiments described. Without wishing to unnecessarily limit the foregoing, the following shall discuss the invention with respect to certain preferred embodiments. The cells of the preferred embodiment of invention can be either eukaryotic cells and/or prokaryotic cells. In a preferred embodiment, the cells are animal cells, and mammalian cells. The cells can be any type of recombinant or non-recombinant prokaryotic cell or eukaryotic cell, including, for example, insect cells, e.g. Sf-9, primate cells, e.g., Vero, mouse, e.g., BHK or C-127, hamster, e.g., CHO, fungal, e.g., Saccharomyces or Scizosaccharomyces, or human, e.g., tumor, osteoblast, fibroblast, and mesenchymal stem cells. Any cells can be grown in the cell-cultivating apparatus in accordance with the preferred embodiment of invention. In particular, cells of choice for the preferred embodiment of invention can be anchorage-dependent or anchorage-independent. Anchorage-dependent cells require a surface on which to grow whereas anchorage-independent cells can grow in liquid suspension. All cell types require adequate oxygen, nutrients, and growth factors to grow.

In another embodiment of the preferred embodiment of invention of cell-cultivating method, which can help to release carbon dioxide during culture and help to stabilize pH during culture are shown in FIG. 15. The method comprises the steps of: providing a growth substrate to receive cells and allow cell adhesion; providing culture medium to submerge the substrate means to provide sufficient nutrient; removing excess culture medium from the substrate means and remain only a thin film of culture medium covering on the substrate means and replace the space originally occupied by culture medium with gaseous phase; allowing the substrate means to stay in the gaseous phase for a period of time; allow a air to flow through the substrate means in order to remove excess carbon dioxide; re-submerging the substrate means with culture medium to provide sufficient nutrient and remove waste from the substrate means; repeat the submerging and emerging step until the purpose of the cell culture is achieved.

### EXAMPLES

The following example is set forth to illustrate an embodiment in accordance with the preferred embodiment of invention, it is by no way limiting nor does this example impose a limitation on the preferred embodiment of invention.

### Example 1

### Distribution of Mouse Hybridoma (OKT3) Cells Anchored on Cell Growth Substrate

Culture OKT3 hybridoma cells with medium (IMDM supplemented with 10% FCS/NaHCO3/PSA 1% /pH=7.4). The medium and culture cells introduce in the preferred embodiment of invention (Tide Bag). Tide Bag was placed in a 37° C. incubator and operated as suggested protocol in the Detail Description section. After culture 97.7 hours, using hematoxylin staining the OKT3 cells to observe the distribution of OKT3 Cells anchored on cell growth substrate.

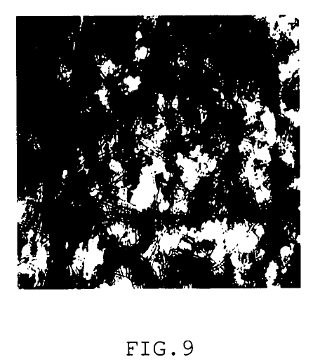

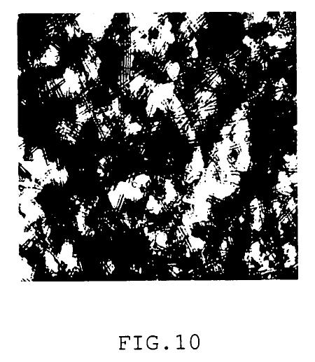

The result of examining under a microscope was shown in FIG. 9. and FIG. 10.

### Example 2

### Compare Glucose Uptake Rate and Glutamine Uptake Rate (GUR ) between the Referred Embodiment of Invention and BelloCell

It takes relatively shorter period of time for the preferred embodiment of invention (Tide Bag) to achieve 97.7 hr GUR:63.8; hence the result is similar to BelloCell Batch 189.5 hr GUR:68.33. It shows that Tide Bag is more efficient in cells preparation than BelloCell (a commerce bioreactor was manufacture by CESCO BIOENGINEERING CO., LTD.)

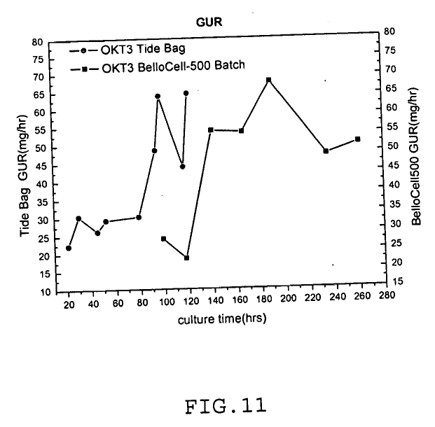

The result of comparing with between the referred embodiment of invention and BelloCell in term of GUR was indicated in FIG. 11

### Example 3

### Compare L/G with pH Value between Present Invention and BelloCell

The preferred embodiment of invention (Tide Bag) is in a closed culture system at beginning, and the longer the culture continues, more carbon dioxide is released from cells, and as a result pH value of culture medium gently decreases. The referred embodiment of invention with continued culture will be more stable in term of pH value.

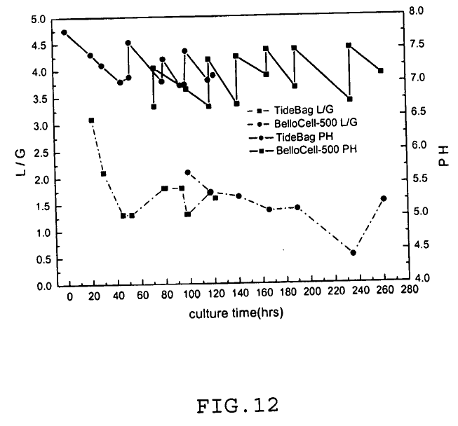

The result of comparing Lactate production to Glucose consumption ratio (L/G) with pH value was shown in FIG. 12. (L/G is an indication of the efficiency of glucose metabolism. Normal range should be around 1.5˜1.8.)

### Example 4

### The Photo of the Preferred Embodiment of Invention

The photo indicates a relative position of the preferred embodiment of invention, which contains cell growth substrate, fresh culture medium bottle connected to an inlet and two outlets for air and conditional medium transfer. A chamber rests on a platform equipped with timing control.

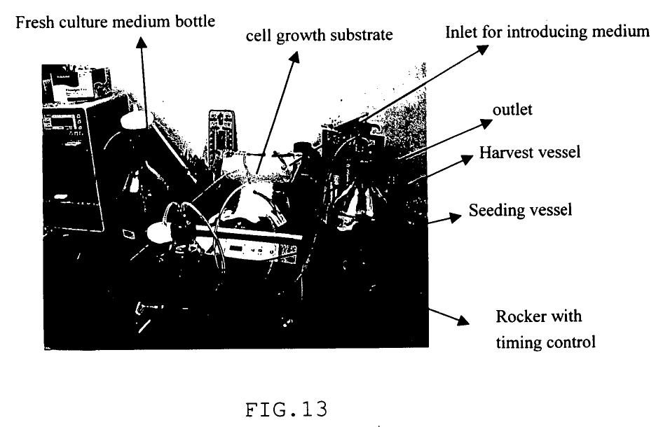

The photo of the preferred embodiment of invention was shown in FIG. 13

1. An apparatus for culturing and preparing cells comprising: at least one chamber, which contains at least one inlet and/or one outlet; one or more cell growth substrate(s) placed in the side(s) of the chamber; a platform, the chamber which is secured on the platform; one or more fulcrums under the platform; a power plant, connected to the fulcrum to move the platform in a seesaw motion or rocking motion in a single or two degree of freedom; a timing controller, which can control the timing for the platform to tilt at one end, to maintain the sloping position for a period of time and then tilt back to the other end and maintain the sloping position for a period of time.

2. The apparatus for culturing and preparing cells according to claim 1, wherein the chamber is a rigid or flexible plastic bag; the bag has a single hollow interior space.

3. The apparatus for culturing and preparing cells according to claim 2, wherein the chamber is a rigid plastic bag.

4. The apparatus for culturing and preparing cells according to claim 2, wherein the chamber is a flexible plastic bag.

5. The apparatus for culturing and preparing cells according to claim 1, wherein the chamber contains at least one inlet and/or one outlet on the surface of the chamber.

6. The apparatus for culturing and preparing cells according claim 5, wherein the chamber contains two inlets and/or two outlets on the surface on the chamber.

7. The apparatus for culturing and preparing cells according to claim 6, wherein one of inlet is for airflow in, the other inlet for introduction of culture medium.

8. The apparatus for culturing and preparing cells according to claim 7, wherein the inlet for airflow in has at least one filter attached to sterilize air passing through the chamber.

9. The apparatus for culturing and preparing cells according to claim 8, wherein the filter is single, dual or multiple.

10. The apparatus for culturing and preparing cells according to claim 6, wherein one outlet is for airflow out, the other outlet is for withdrawing the conditioned culture medium.

11. The apparatus for culturing and preparing cells according to claim 10, wherein the outlet for airflow out has at least one filter attached to sterilize the air passing out of the chamber.

12. The apparatus for culturing and preparing cells according to claim 10, wherein the outlet for withdrawing the conditioned culture medium is further adapted to extend its tubing into the bottom of the chamber.

13. The apparatus for culturing and preparing cells according to claim 11, wherein the filter is single, dual or multiple.

14. The apparatus for culturing and preparing cells according to claim 5, wherein the chamber contains an opening tract closable with a cap.

15. The apparatus for culturing and preparing cells according to claim 14, wherein there is an opening for air permission and air exchange.

16. The apparatus for culturing and preparing cells according to claim 14, wherein there is an opening for introducing cells, culture mediums and withdrawal of conditioned culture mediums.

17. The apparatus for culturing and preparing cells according to claim 1, wherein the growth substrate comprises of a polymer folding into a three-dimensional structure.

18. The apparatus for culturing and preparing cells according to claim 17, wherein the polymer is made of a porous and/or non-porous material, high-molecular material, ceramics, fibers, and non-woven sheets or woven sheets.

19. The apparatus for culturing and preparing cells according to claim 18, wherein the polymer is a porous material, high-molecular material, ceramics, fibers and woven sheets.

20. The apparatus for culturing and preparing cells according to claim 1, wherein the platform can be tilted in a seesaw motion at a degree from 0 to 180.

21. The apparatus for culturing and preparing cells according to claim 1, wherein the fulcrums can be one or more in order to control the platform to tilt at a single or two degree of freedom.

22. The apparatus for culturing and preparing cells according to claim 1, wherein the power plant is a motor.

23. The apparatus for culturing and preparing cells according to claim 1, wherein the power plant is a hydraulic cylinder.

24. The apparatus for culturing and preparing cells according to claim 1, wherein the power plant is an air cylinder.

25. A method for culturing and preparing of cells, comprising the steps of: (a) preparing a sterile chamber which has a single hollow interior volume, and has cell growth substrate(s) mounted on one or more terminal sides in the chamber; (b) introducing air containing oxygen using the inlet to partially inflate the said chamber (c) introducing cell culture medium using the inlet(s); (d) placing cells suspension into the chamber to distribute cells on the cell growth substrate; (e) securing the chamber on the platform; (f) placing a fulcrum under the platform; (g) introducing air containing oxygen using the inlet(s), and opening the outlet(s), to inflate chamber with air continual pass through chamber; (h) connecting a power supply, connected to the fulcrum to move the platform in a seesaw or rocking motion; (i) setting up a timing control in order to keep one of cell culture substrate be submerged or exposed for a period of time in order to allow cells to anchor on the cell growth substrate or be embedded in the cell growth substrate, and to keep the seesaw or rocking motion until cells are anchored on growth substrate and/or embedded the porous growth substrate; (j) setting up a timing control in order to keep one of cell culture substrate be submerged or exposed for a period of time in order to complete the nutrient exchange or control nutrient supply for both gaseous and liquid phase; (k) keeping moving the platform in a seesaw or rocking motion, wherein the cell growth substrate being submerged at one side, whereby the necessary carbon dioxide and nutrition being transferred/mixed; and thin film of culture medium exposed on cell growth substrate at the other side, whereby oxygen is received through a thin film medium interface without directly contacting air during the see-saw or rocking motion; (l) periodically removing conditioned culture medium, wherein containing cellular products secreted by cells ; (m) periodically repeating step (j) to (l) for maintaining suitable continual cell culture condition during the cultured period.

26. The method for culturing and preparing of cells according to claim 25, wherein the cells to be cultured are anchorage-dependent cells attached on growth substrate.

27. The method for culturing and preparing of cells according to claim 25, wherein the cells to be cultured are anchorage-independent cells embedded in the porous growth substrate.

28. The method for culturing and preparing of cells according to claim 25, wherein the cells are selected from the group consisting of eukaryotic, prokaryotic, mammalian, animal, human, bacterial, and fungal cells.

29. The method for culturing and preparing of cells according to claim 25, wherein the disposing the chamber is in the range of 0 sec to 24 hour for cells to anchor or embed on/in the growth substrate, depending on the different variety and/or density of cells.

30. The method for culturing and preparing of cells according to claim 25, wherein the disposing the chamber is in the range of 0 sec to 4 hour for cells to anchor or embed on/in the growth substrate, depending on the different variety and/or density of cells.

31. The method for culturing and preparing of cells according to claim 25, wherein placing the cell growth substrate is the chamber.

32. The method for culturing and preparing of cells according to claim 25, wherein the timing control of exposure and/or submerge cell growth substrate is in the range of 0 to 24 hour, depending on the different variety and/or density of cells.

33. The method for culturing and preparing of cells according to claim 25, wherein the timing control of exposure cell growth substrate is in the range of 0.5 to 12 hour, depending on the different variety and/or density of cells.

34. The method for culturing and preparing of cells according to claim 25, wherein the timing control of exposure cell growth substrate is in the range of 0.5 to 2 hour, depending on the different variety and/or density of cells.

35. The method for culturing and preparing of cells according to claim 25, wherein the timing control of exposure cell growth substrate is in the range of 0 to 1 hour, depending on the different variety and/or density of cells.

36. The method for culturing and preparing of cells according to claim 25, wherein the growth substrate comprises of a polymer folding into a three-dimensional structure.

37. The method for culturing and preparing of cells according to claim 36, wherein the polymer is made of a porous and/or non-porous material, high-molecular material, ceramics, fibers, and non-woven sheets or woven sheets.

38. The method for culturing and preparing cells according to claim 37, wherein the polymer is a porous material, high-molecular material, ceramics, fibers and woven sheets.
